(19) United States
(12) Patent Application Publication
(10) Pub. No.: US 2010/0160949 A1
(43) Pub. Date: Jun. 24, 2010

(54) ANEURYSM EMBOLIZATION DEVICE AND OPERATION METHOD THEREOF

(76) Inventor: Norikata TAKUMA, Kokubunji City (JP)

Correspondence Address: JORDAN AND HAMBURG LLP, 122 EAST 42ND STREET, SUITE 4000, NEW YORK, NY, 10168, US

(21) Appl. No.: 12/574,127

(22) Filed: Oct. 6, 2009

(30) Foreign Application Priority Data
Dec. 22, 2008 (JP) 2008-324992

(51) Int. Cl. A61M 29/00

(52) U.S. Cl. 606/195

(57) ABSTRACT

An aneurysm embolization device includes at least: a sac-shaped balloon dome part which is inserted into the aneurysm, then expanded and left therein; and a balloon plane part which is provided at an opening of the balloon dome part and covers a mouth part of the aneurysm. The balloon plane part has a hole communicating with the inside of the balloon dome part. The aneurysm embolization device thus constituted is housed into a lumen of a protective outer sheath member formed of a flexible elongated-body having the lumen therein. A tip part of the protective outer sheath member is placed at a desired position, and the aneurysm embolization device is ejected from the tip part of the protective outer sheath member. The aneurysm embolization device thus ejected includes the balloon dome part placed at a desired position and the balloon plane part deploying to cover the desired position.

### BACKGROUND OF THE INVENTION

1. Field of the Invention

The present invention relates to an aneurysm embolization device for treating an aneurysm in a blood vessel by occluding the aneurysm, and an operation method thereof. Specifically, the present invention relates to techniques for treating an aneurysm according to the site, the size, the shape, and the like of the aneurysm.

2. Description of the Related Art

An aneurysm is local dilatation of a blood vessel due to weakening of the vascular wall, and usually has a size of 3 mm to 4 mm, or sometimes of 15 mm or larger in diameter. If untreated, an aneurysm can increase in size, and eventually ruptures. The rupturing of the aneurysm is likely to cause fatal bleeding: for example, bleeding onto the brain surface (subarachnoid hemorrhage). A clipping method and intravascular surgical techniques are known for treating such aneurysms.

The clipping method is a surgical technique that blocks blood from flowing into an aneurysm and eliminates the aneurysm from blood flow with a pinching member called a clip, which is made of an elastic, corrosion-resisting material such as titanium. Specifically, in the method, the aneurysm is exposed by craniotomy, and a neck part (the base) of the aneurysm is pinched with the clip for the blockage and elimination in order to prevent the aneurysm from rupturing (see, for example, Japanese Examined Patent Application Publication No. Hei 07-004389).

Meanwhile, intravascular surgical techniques include balloon placement, coagulant injection, coil embolization and stent placement techniques.

The balloon placement is a technique to occlude an aneurysm with a balloon using a catheter. The balloon, which is made of latex or silicone rubber and connected to a tip of the catheter, is guided to an aneurysm, inserted and expanded in the aneurysm. Then, the balloon is detached from the catheter. In other words, this is a technique to place a balloon in an aneurysm to occlude a sac and neck of the aneurysm while maintaining blood flow in a parent vessel (artery) (see, for example, Japanese Patent Translation Publication No. 2004-520881 and “Nokekkan-nai chiryo no Do's & Don'ts (Do's & Don'ts in intracerebrovascular treatment)” pp. 1-4, published by Igaku-Shoin Ltd.).

The coagulant injection is a technique to inject a liquid coagulating substance (i.e., liquid embolic material) inside an aneurysm (see, for example, Japanese Unexamined Patent Application Publication No. Hei 06-107549 and “Nokekkan-nai chiryo no Do's & Don'ts (Do's & Don'ts in intracerebrovascular treatment)” pp. 1-4, published by Igaku-Shoin Ltd.).

The coil embolization is a technique in which: a catheter is percutaneously introduced into a blood vessel from, for example, a femoral region; a tip of the catheter is moved to an aneurysm site under radiographic guidance; and an embolus, such as a platinum coil, is supplied from a lumen formed in the catheter and fills (packs) the inside of the aneurysm (see, for example, Japanese Patent Application Publication No. 2003-070794 and “Nokekkan-nai chiryo no Do's & Don'ts (Do's & Don'ts in intracerebrovascular treatment)” pp. 1-4, published by Igaku-Shoin Ltd.).

The stent placement is a technique to block blood from flowing into an aneurysm from a parent vessel with a tubular member called a stent. The stent is left at a mouth part of the aneurysm, and radially expands at a narrow part of the vessel from the inside of the blood vessel to cover the aneurysm (see, for example, Japanese Patent Application Publication No. 2004-33535).

There are also other techniques: to inject a coagulating substance into an aneurysm through slits arranged in a peripheral body part of a stent; and to segregate an aneurysm in the blood vessel from blood flow by covering the mouth part of the aneurysm using the stent as a scaffold (see, for example, Japanese Patent No. 4057318).

All of such aneurysm treatments provide reduction in the blood flowing into the aneurysm and result in blockage of blood flow and formation of a clot. In other words, these techniques can treat aneurysms based on pathophysiological characteristics of blood in which: blood clots when its flow stops; abnormal blood vessel inner wall or contact between blood and foreign substances causes formation of blood clot; and the like.

However, in the clipping method as represented by the invention described in Japanese Examined Patent Application Publication No. Hei 07-004389, it is difficult to pinch a neck part of an aneurysm with a clip when the neck part is wide (i.e., when the mouth part of the aneurysm has a large diameter). In addition, a lot of preparation tasks are required for the craniotomy because it is a major surgery as compared with an intravascular surgery. Furthermore, the surgery may compress or damage the brain tissue.

In the balloon placement technique as represented by the invention described in Japanese Patent Translation Publication No. 2004-520881, a balloon which is tied to a catheter using a rubber string is prepared. Then, the balloon is detached from the catheter or a joint between the balloon and the catheter is separated using a high-frequency electric current so that the balloon may not be deflated. However, it is very difficult to keep the balloon from deflating after the balloon is detached from the catheter, and it is also required to keep watch on the balloon not to deflate even after the separation. In addition, the shape of the neck part of aneurysms greatly differs one from the others, but the shape of balloons is virtually the same. As a result, the balloon is not able to occlude the aneurysm completely because the shape of the neck part of aneurysm and that of the balloon do not always match (i.e., the balloon is not able to correspond to various shapes of aneurysms).

In the coagulant injection technique as represented by the invention described in Japanese Unexamined Patent Application Publication No. Hei 06-107549, selection of a liquid substance to fill an aneurysm is difficult, because the liquid substance must be liquid until filled into the aneurysm and must promptly coagulate right after filled into the aneurysm. In other words, if the coagulation rate of the liquid substance is too fast, the substance coagulates in a catheter before it is filled into an aneurysm. Meanwhile, if the coagulation rate of the liquid substance is too slow, the liquid substance filled in the aneurysm would flow out into blood vessel.

Besides, the coil embolization technique as represented by the invention described in Japanese Patent Application Publication No. 2003-070794 overcomes the problems of the coagulant injection and balloon placement techniques. However, packing of an aneurysm with a coil is difficult in the coil embolization technique, and there is also a risk of bleeding by breaking the aneurysm wall during a process of the operation. Furthermore, there are risks that a part of the coil may stick out into the inside of the parent vessel, or that the coil packed in the aneurysm may escape into the parent vessel if the neck part of the aneurysm is wide. Still furthermore, even if the coil is packed into the aneurysm completely, approximately half a space in the aneurysm remains unfilled. The space may allow blood to flow in the aneurysm, and thus embolization could be failed in some cases.

In addition, the stent placement as represented by the inventions described in Japanese Patent Application Publication No. 2004-33535 and Japanese Patent No. 4057318 has a risk to block a way in other necessary branched blood vessel as well as the aneurysm if the blood vessel is branched around the site of the aneurysm.

### SUMMARY OF THE INVENTION

The present invention addresses the problems discussed above. An object of the present invention is to provide an aneurysm embolization device capable of occluding an aneurysm readily and reliably in an appropriate manner according to the site, the shape of a neck part, and the size of the aneurysm.

Another object of the present invention is to provide a method for operating an aneurysm embolization device, using the above-mentioned aneurysm embolization device, the method being capable of occluding an aneurysm readily and reliably.

An aneurysm embolization device according to a first aspect of the present invention is an aneurysm embolization device for treating an aneurysm in a blood vessel by occluding the aneurysm. The aneurysm embolization device includes at least a sac-shaped balloon dome part which is inserted into the aneurysm, then expanded and left therein; and a balloon plane part which is provided at an opening of the balloon dome part and covers a mouth part of the aneurysm; in which the balloon plane part has a hole communicating with the inside of the balloon dome part.

An aneurysm embolization device according to a second aspect of the present invention is the aneurysm embolization device according to the first aspect, in which the balloon plane part further has an anchor member (stent) which is expanded and left in the blood vessel.

An aneurysm embolization device according to a third aspect of the present invention is the aneurysm embolization device according to the second aspect, in which the anchor member is attached to at least one end of the balloon plane part.

A method for operating an aneurysm embolization device according to a fourth aspect of the present invention is a method for operating an aneurysm embolization device for treating an aneurysm in a blood vessel by occluding the aneurysm, the aneurysm embolization device including at least: a sac-shaped balloon dome part which is inserted into the aneurysm, then expanded and left therein; and a balloon plane part which is provided at an opening of the balloon dome part and covers a mouth part of the aneurysm, the balloon plane part having a hole communicating with the inside of the balloon dome part. The method includes the successive steps of: α1) housing the aneurysm embolization device into a lumen of a protective outer sheath member formed of a flexible elongated-body having the lumen therein; α2) manipulating the protective outer sheath member to place a tip part of the protective outer sheath member at a desired position where the aneurysm embolization device is intended to be left; and α3) ejecting the aneurysm embolization device from the tip part of the protective outer sheath member, placing the balloon dome part at the desired position, and then, deploying the balloon plane part to cover the desired position.

A method for operating an aneurysm embolization device according to a fifth aspect of the present invention is the method for operating an aneurysm embolization device according to the fourth aspect, in which, in the step α3, the aneurysm embolization device is ejected by pressure of a liquid which is supplied through the inside of the lumen of the protective outer sheath member.

A method for operating an aneurysm embolization device according to a sixth aspect of the present invention is a method for operating an aneurysm embolization device for treating an aneurysm in a blood vessel by occluding the aneurysm, the aneurysm embolization device including at least: a sac-shaped balloon dome part which is inserted into the aneurysm, then expanded and left therein; and a balloon plane part which is provided at an opening of the balloon dome part and covers a mouth part of the aneurysm, the balloon plane part having a hole communicating with the inside of the balloon dome part, the balloon plane part further including an anchor member (stent) which is expanded and left in the blood vessel and an expansion balloon which supports the expansion of the anchor member. The method includes the successive steps of: β1) housing the aneurysm embolization device into a lumen of a protective outer sheath member formed of a flexible elongated-body having the lumen therein; β2) manipulating the protective outer sheath member to place a tip part of the protective outer sheath member at a desired position where the aneurysm embolization device is intended to be left; β3) ejecting the aneurysm embolization device from the tip part of the protective outer sheath member, expanding the anchor member by inflating the expansion balloon, placing the balloon dome part at the desired position, and then, deploying the balloon plane part to cover the desired position; and β4) deflating the expansion balloon and then sliding back the expansion balloon from the inside of the anchor member.

The aneurysm embolization device of the present invention includes a sac-shaped balloon dome part which is inserted into an aneurysm in a blood vessel, then expanded and left therein; and a balloon plane part which is provided at an opening of the balloon dome part and has a hole communicating with the inside of the balloon dome part. Accordingly, when the aneurysm embolization device is placed so that the balloon dome part can be inserted into the aneurysm, the balloon plane part is left on a mouth part of the aneurysm and covers the mouth part of the aneurysm and a desired area around the aneurysm regardless of the shape and size of the mouth part of the aneurysm. Moreover, the hole of the balloon plane part enables blood streaming in a parent vessel to flow into the balloon dome part. As a result, the stream of the blood flowed into the balloon dome part is repressed by covering and closing the aneurysm with the balloon plane part, and thus, the blood in the balloon dome part forms a clot to occlude the aneurysm. In addition, because of the clot formation by the blood in the balloon dome part, the balloon dome part prevents the aneurysm embolization device from being swept away by the blood flow, thus functioning as an anchor member to aid the remaining of the device at the site of aneurysm.

Thus, the present invention can provide an aneurysm embolization device capable of occluding an aneurysm readily and reliably in an appropriate manner according to the site, the shape of a neck part, and the size of the aneurysm.

Meanwhile, in the method for operating an aneurysm embolization device of the present invention, the aneurysm embolization device including a balloon dome part and a balloon plane part is housed into a tip part of a lumen of a protective outer sheath member formed of a flexible elongated-body having the lumen therein. Then, the protective outer sheath member is manipulated to place the tip part of the protective outer sheath member at a desired position where the aneurysm embolization device is intended to be left. Subsequently, the aneurysm embolization device is ejected from the tip part, and the balloon dome part is placed at the desired position, and the balloon plane part is deployed to cover the desired position.

Thus, the present invention can provide a method for operating an aneurysm embolization device, using the above-mentioned aneurysm embolization device, the method capable of occluding an aneurysm readily and reliably.

### BRIEF DESCRIPTION OF THE DRAWINGS

FIGS. 1A and 1B are drawings showing a structure of an aneurysm embolization device according to a first embodiment of the present invention. FIG. 1A is a perspective view showing the entire structure. FIG. 1B is a longitudinal sectional view taken along the line I-I in FIG. 1A.

FIG. 2 is a longitudinal sectional view of an aneurysm embolization device according to a second embodiment of the present invention.

FIG. 3 is a longitudinal sectional view of another aneurysm embolization device according to the second embodiment of the present invention.

FIG. 4 is a longitudinal sectional view of another aneurysm embolization device according to the second embodiment of the present invention.

FIG. 5 is a longitudinal sectional view of an aneurysm embolization device according to a third embodiment of the present invention.

FIG. 6 is a longitudinal sectional view of an aneurysm embolization device according to a fourth embodiment of the present invention.

FIG. 7 is a longitudinal sectional view of another aneurysm embolization device according to the fourth embodiment of the present invention.

FIGS. 8A to 8C are sectional process drawings for sequentially illustrating a method for operating the aneurysm embolization device according to the fourth embodiment of the present invention shown in FIG. 6.

FIGS. 9A to 9C are sectional process drawings for sequentially illustrating a method for operating the aneurysm embolization device according to the fourth embodiment of the present invention shown in FIG. 6.

FIGS. 10A and 10B are sectional process drawings for sequentially illustrating a method for operating the aneurysm embolization device according to the fourth embodiment of the present invention shown in FIG. 6.

FIGS. 11A and 11B are sectional process drawings for sequentially illustrating a method for operating the aneurysm embolization device according to the third embodiment of the present invention shown in FIG. 5.

FIGS. 12A and 12B are sectional process drawings for sequentially illustrating a method for operating the aneurysm embolization device according to the third embodiment of the present invention shown in FIG. 5.

### DETAILED DESCRIPTION OF THE PREFERRED EMBODIMENTS

Hereinafter, examples of embodiments of the present invention will be described with reference to the drawings.

Note that the following embodiments of the present invention have various technological restrictions because these are specific preferable examples of the present invention. However, the scope of the present invention is not limited to these embodiments unless it is otherwise particularly described to be limited in the following explanations.

An aneurysm embolization device intended in the present invention is an aneurysm embolization device to treat an aneurysm in a blood vessel by occluding the aneurysm readily and reliably in an appropriate manner according to the site, the shape of a neck part, and the size of the aneurysm.

### Embodiment 1

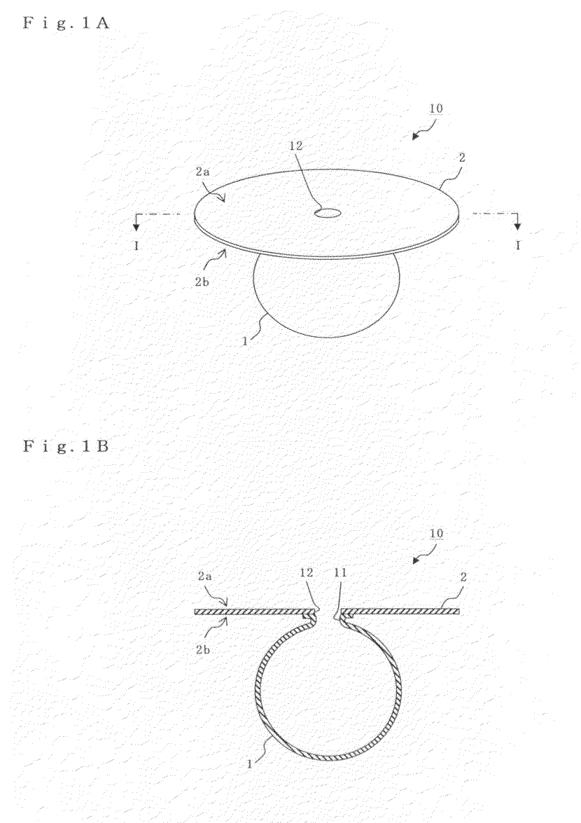

FIGS. 1A and 1B are diagram showing a structure of an aneurysm embolization device according to a first embodiment of the present invention. FIG. 1A is a perspective view showing the entire structure, and FIG. 1B is a longitudinal sectional view taken along the line I-I in FIG. 1A.

As shown in FIGS. 1A and 1B, the aneurysm embolization device 10 of this embodiment includes a balloon dome part 1 and a balloon plane part 2. Incidentally, FIGS. 1 A and 1B are shown schematically with slight exaggeration to facilitate the understanding of functions of the aneurysm embolization device of the present invention (including functions of both the balloon dome part and the balloon plane part).

The balloon dome part 1 is a sac body, which is inserted into an aneurysm, expanded by influx of blood, and left in the aneurysm. This balloon dome part 1 is made of a flexible, stretchable and thin biocompatible material using, for example, latex or silicone. Additionally, it is desirable that the entire outside diameter of the balloon dome part 1 be almost equal to or slightly smaller than the inside diameter of the aneurysm.

The balloon plane part 2 is a sheet body which is provided at an opening 11 of the balloon dome part 1 and covers a mouth part of the aneurysm. This balloon plane part 2 is made of a flexible thin biocompatible material. In FIG. 1A, the balloon plane part 2 is indicated as a round shape, but it is not limited to this shape. The balloon plane part 2 may be oval, rectangular, or the like, and can be arbitrarily designed or selected depending on the shape and size of a mouth part of the aneurysm to be occluded. Accordingly, it is desirable that size of the balloon plane part 2 be larger than the diameter of the mouth part of the aneurysm.

In this aneurysm-mouth-part closing film 2, it is desirable that one surface 2a of the film be formed from a hard amorphous film such as diamond-like carbon, and that another surface 2b on the opposite side from the one surface 2a have a rough surface like cilia. Such a structure makes clot formation hardly occur on the one surface 2a side of the aneurysm-mouth-part closing film 2; meanwhile, clot formation is likely to occur on the other surface 2b side of the film 2. Therefore, the aneurysm embolization device 10 is placed in a manner that the one surface 2a of the aneurysm-mouth-part closing film 2 faces a parent vessel, and that the other surface 2b faces an aneurysm.

The balloon plane part 2 has a hole 12 communicating with the inside of the balloon dome part 1. As long as the hole 12 allows blood to flow into the balloon dome part 1, the shape of the hole 12 is not particularly limited. The balloon plane part 2 may have an opening from the beginning, or a slit hole opened by flux of blood. In FIGS. 1A and 1B, the hole 12 is shown as a circular opening which is opened originally. Although the hole size of the hole 12 in diameter is not specified particularly, the size is large enough to allow blood to flow into the balloon dome part 1, and is small enough to prevent clots solidified in the balloon dome part 1 from flowing out.

As long as the balloon dome part 1 and the aneurysm-mouth-part closing film 2 are integrated each other, other features are not particularly limited. For example, these parts may be integrally formed using the same material, or may be formed separately and then integrated together. In addition, integration of the aneurysm-mouth-part closing film 2 to the opening 11 of the balloon dome part 1 may be accomplished by joining the dome part 1 to the film 2 using an adhesive, or may be accomplished by suturing or fusion bonding.

According to the aneurysm embolization device 10 with the above-described constitution, when the aneurysm embolization device 10 is placed so that the balloon dome part 1 can be inserted into an aneurysm, the balloon plane part 2 on the one surface 2a side is pushed by blood flow streaming in a parent vessel, meanwhile, the other surface 2b is pressed against the inner blood vessel wall. Thus, the balloon plane part 2 is left on a mouth part of the aneurysm. Accordingly, the balloon plane part 2 covers the mouth part of the aneurysm and a desired area around the aneurysm from inside of the parent vessel. Furthermore, when the balloon plane part 2 covers the mouth part of the aneurysm, blood streaming in the parent vessel flows into the balloon dome part 1 through the hole 12 arranged in the balloon plane part 2, expands the balloon dome part 1, and fills the inside of the balloon dome part 1. Then, the stream of the blood flowed into the balloon dome part 1 is repressed, and the blood in the balloon dome part 1 forms clot to occlude the aneurysm. Therefore, it is possible to occlude aneurysm readily and reliably in an appropriate manner according to the shape of a neck part of the aneurysm and the size of the aneurysm by using the aneurysm embolization device 10 with a very simple constitution.

In this occlusion process, when the size of the blood clot formed in the balloon dome part 1 is larger than that of the mouth part of the aneurysm, the clotted blood is locked at an edge of the aneurysm mouth part. This makes it possible to prevent the balloon dome part 1 from being easily detached from the aneurysm for a long period. Furthermore, it is desired that surface treatment be performed on the inner and outer surfaces of the balloon dome part 1 to stimulate clot formation.

### Embodiment 2

In the present invention, an aneurysm embolization device can be left in a blood vessel more stably. In other words, a second embodiment of the present invention is different from the above-described first embodiment in that the second embodiment includes means for stabilizing the balloon plane part 2 in a blood vessel.

Note that, in other embodiments to be described below, description will be given mainly of aspects which are different from those in the above-described first embodiment of the present invention. Therefore, the same letters or numerals are given to constituents which are similar to those in the first embodiment, and explanations thereof are omitted. The constituents represented by the same letters or numerals will be the same unless it is otherwise particularly described.

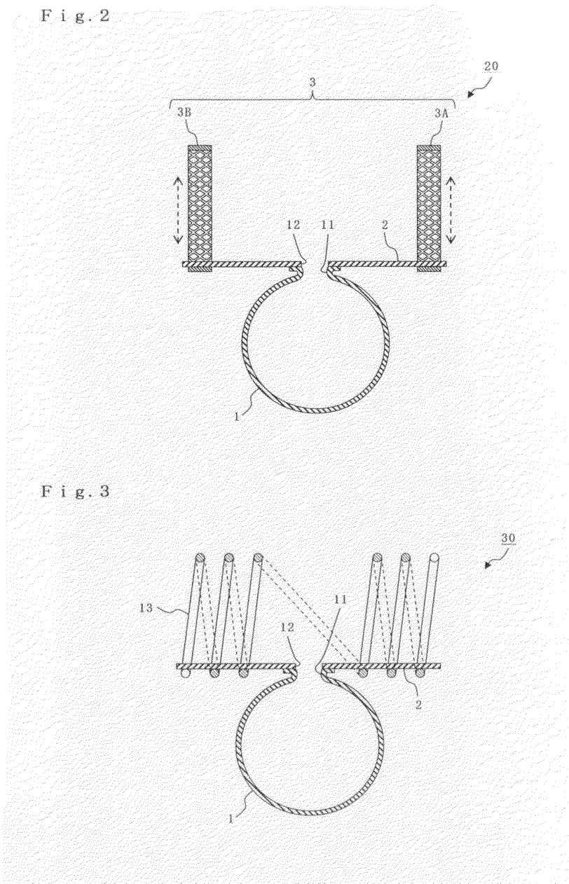

An aneurysm embolization device of this embodiment including mean for stabilizing the device in a blood vessel can be exemplified as shown in FIG. 2. The aneurysm embolization device shown here has a beneficial effect on usage for a type of aneurysm which is exserted from a side surface of a blood vessel, for example.

As shown in FIG. 2, an aneurysm embolization device 20 of this embodiment includes the balloon dome part 1 and the balloon plane part 2. Additionally, the balloon plane part 2 further has an anchor member 3 which is expanded and left in a blood vessel. The anchor member 3 can be stabilized in a blood vessel without displacement, and can be, for example, an approximately tubular stent with a net-like structure. This anchor member (hereinafter, referred to as “stent”) 3 can be formed using a material for medical application such as stainless steel, tantalum, cobalt base alloy and nickel-titanium alloy (nitinol).

Furthermore, as long as the stent 3 is attached to the balloon plane part 2 in an integrated manner, the way of the attachment is not particularly limited. Therefore, for example, these components may be jointed using an adhesive, or the stent 3 may be rolled up, pinched or sutured by the balloon plane part 2.

In FIG. 2, the stents 3 is constituted of two approximately tubular (annular) body cells 3A and 3B, which are separated from each other with a hole 12 in between and attached to the respective opposed ends of the balloon plane part 2. The cells can radially expand toward the blood vessel wall as shown with dotted arrows in the figure. The stent 3 (3A and 3B) can be stabilized in a blood vessel without displacement by pressing the stent 3 to the blood vessel wall.

According to the aneurysm embolization device 20 with the above-described constitution, the balloon plane part 2 can be reliably placed adjacent to the mouth part of the aneurysm by expanding the stent 3 in a blood vessel and pressing the stent 3 to the blood vessel wall. Thus, the aneurysm embolization device 20 can be stably left at the mouth part of the aneurysm.

It is also desirable that the stent 3 (3A and 3B) of the above-described second embodiment be attached to at least one end of the balloon plane part 2. The term “one end” refers to a side of a blood vessel which is closer to the heart than an aneurysm, in other words, an upstream side of a blood flow. Therefore, only any one of the two approximately tubular (annular) body cells 3A and 3B shown in FIG. 2 should be provided as the stent 3.

As mentioned above, despite such a simple structure, another end of the balloon plane part 2 deploys along the bloodstream and can ensure stable placement of the aneurysm embolization device in the blood vessel without displacement.

Moreover, in this embodiment, the means for stabilizing the balloon plane part 2 to a blood vessel is not limited to the above-described approximately tubular stent. For example, as shown in FIG. 3, a coiled stent 13 can be also used. This stent 13 can also be formed using a material for medical application such as stainless steel, tantalum, cobalt alloy and nickel-titanium alloy (nitinol).

In FIG. 3, the stent 13 is constituted with a spiral and approximately tubular body with more space around the hole 12. This stent 13 can also be stabilized in a blood vessel without displacement by pressing the stent 13 to the blood vessel wall.

According to the aneurysm embolization device 30 with the above-described constitution, the balloon plane part 2 can be reliably placed adjacent to the mouth part of the aneurysm by expanding the stent 13 in a blood vessel and pressing the stent 13 to the blood vessel wall. Thus, the aneurysm embolization device 30 can be stably left at the mouth part of the aneurysm.

It is also desirable that the stent 13 of the above-described embodiment be attached to at least one end of the balloon plane part 2.

Furthermore, when the present invention includes the means for stabilizing the balloon plane part 2 in a blood vessel through the radial expansion, it is desirable that the means more effectively expand radially. In other words, the above-mentioned approximately tubular stent 3 shown in the second embodiment should autonomously expand with its elastic force at a desirable position. However, if not, and if the stent is sought to expand at a desirable position, additional means is needed to support the expansion of the means for stabilizing the balloon plane part 2 in a blood vessel. Therefore, in this embodiment, the expansion supporting means is further provided.

The aneurysm embolization device including the expansion supporting means of this embodiment can be exemplified in FIG. 4.

As shown in FIG. 4, the aneurysm embolization device of this embodiment further includes an expansion balloon 4 which is expandable inside the stent 3. The expansion balloon 4 can push and expand the stent 3 from the inside of the stent 13 by inflating, and can be, for example, a flexible and stretchable bag made of latex, silicone, or the like.

In FIG. 4, the expansion balloon 4 is constituted so that the balloon 4 is inserted and placed into the stent 3 constituted of the approximately tubular body cells 3A and 3B, and that the balloon 4 can be evenly inflated by slowly introducing a liquid, such as saline, through a liquid-supplying tube 14 thereinto.

According to the aneurysm embolization device with the above-described constitution, radial expansion of the stent 3 in a blood vessel is supported, and the balloon plane part 2 can be stabilized in the blood vessel efficiently and effectively.

### Embodiment 3

In the present invention, the influx of blood into the balloon dome part 1 can be ensured. In other words, the diameter of the opening 11 is a size that at least allows influx of blood from the hole 12 of the balloon plane part 2 in the above-described balloon dome part 1 of the first embodiment. However, if the diameter of the opening 11 is small, the opening 11 may be twisted during operation of the aneurysm embolization device; as a result, there is a risk to block influx of blood into the balloon dome part 1. Therefore, a third embodiment of the present invention is different from the above-described first embodiment in the structure of an opening 11 of a balloon dome part 1.

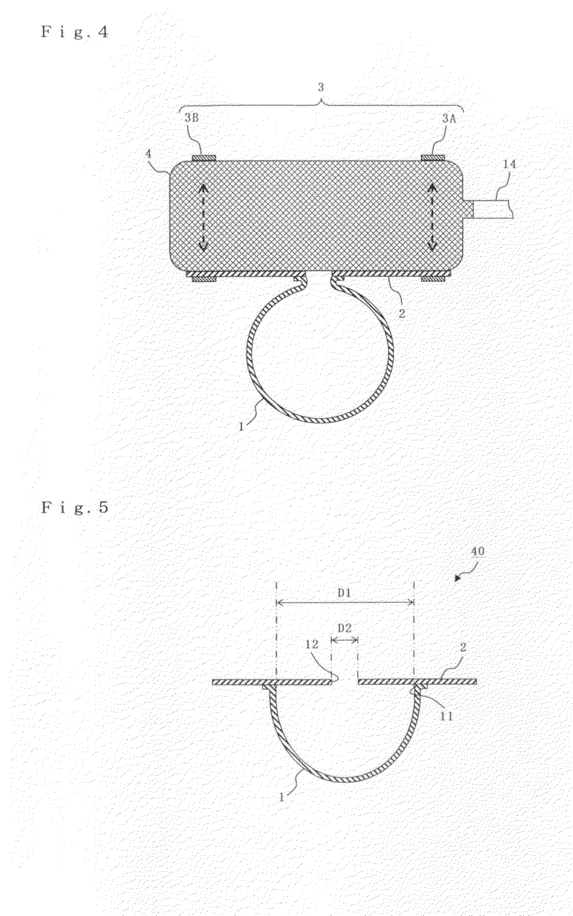

An aneurysm embolization device of this embodiment can be exemplified in FIG. 5.

As shown in FIG. 5, an aneurysm embolization device 40 of this embodiment includes the balloon dome part 1 and the balloon plane part 2. The balloon plane part 2 further has the hole 12 communicating with the inside of the balloon dome part 1. The opening 11 of the dome part 1 has a diameter D1 larger than a diameter D2 of the hole 12 of the balloon plane part 2 (D1>D2).

According to the aneurysm embolization device 40 with the above-described constitution, the opening 11 of the balloon dome part 1 can be protected from excess distortion which occurs during operation. Thus, blood is able to flow into the balloon dome part 1 through the hole 12 of the balloon plane part 2 via the opening 11 of the balloon dome part 1 reliably. Furthermore, the aneurysm embolization device 40 according to this embodiment can adequately deal with an aneurysm, for example, which has a hemispherical shape with such a wide neck part that it is difficult to clip (to pinch) the neck part or which has such a shape that packed coils easily escape from the aneurysm. Therefore, it is preferable to use the aneurysm embolization device 40 of this embodiment as a fundamental tool for such a type of aneurysm which is exserted from a side surface of a blood vessel.

### Embodiment 4

An aneurysm embolization device of a fourth embodiment of the present invention is the aneurysm embolization device of the third embodiment which can be also left in a blood vessel more stably as in the above-described aneurysm embolization device illustrated in the second embodiment. In other words, the aneurysm embolization device of the fourth embodiment further includes an anchor member (stent).

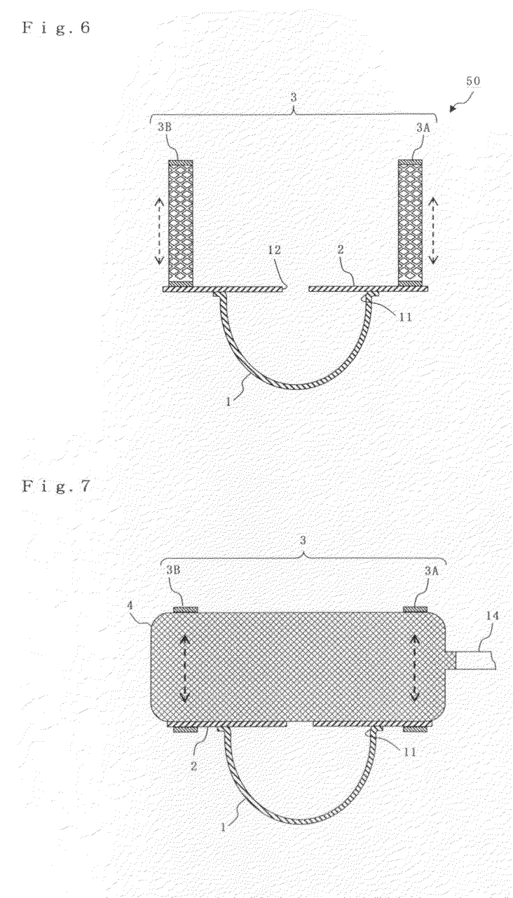

The aneurysm embolization device of this embodiment can be exemplified in FIG. 6.

As shown in FIG. 6, an aneurysm embolization device 50 of this embodiment includes the balloon dome part 1 and the balloon plane part 2. Additionally, the balloon plane part 2 further has an anchor member 3 which is expanded and left in a blood vessel. As in the above-described second embodiment, the anchor member 3 of the fourth embodiment should be stabilized in a blood vessel without displacement, and can be, for example, an approximately tubular stent with a net-like structure. This anchor member (hereinafter, referred to as “stent”) 3 can also be formed using a material for medical application such as stainless steel, tantalum, cobalt alloy and nickel-titanium alloy (nitinol).

Furthermore, as long as the stent 3 is attached to the balloon plane part 2 in an integrated manner, the way of the attachment is not particularly limited. For example, these components may be jointed using an adhesive, or the stent 3 may be rolled up, pinched or sutured by the balloon plane part 2.

In FIG. 6, the stent 3 is constituted of the two approximately tubular (annular) body cells 3A and 3B, which are separated from each other with the hole 12 in between and attached to the respective ends of the balloon plane part 2. The cells can radially expand as shown with dotted arrows in the figure. The stent 3 (3A and 3B) can be stabilized in a blood vessel without displacement by pressing the stent 3 to the blood vessel wall.

According to the aneurysm embolization device 50 with the above-described constitution, the balloon plane part 2 can be reliably placed adjacent to the mouth part of the aneurysm by expanding the stent 3 in a blood vessel and pressing the stent 3 to the blood vessel wall. Thus, the aneurysm embolization device 50 can be stably left at the mouth part of the aneurysm.

It is also desirable that the stent 3 (3A and 3B) of the above-described fourth embodiment be attached to at least only one end of the balloon plane part 2. The term “one end” refers to a side of a blood vessel which is closer to the heart than an aneurysm, in other words, an upstream side of a blood flow. Therefore, only any one of the two approximately tubular (annular) body cells 3A and 3B shown in FIG. 6 should be provided as the stent 3.

As mentioned above, despite such a simple structure, another end of the balloon plane part 2 deploys along the bloodstream and can ensure stable placement of the aneurysm embolization device in the blood vessel without displacement.

Moreover, in this embodiment, the means for stabilizing the balloon plane part 2 to a blood vessel is not limited to the above-described approximately tubular stent. Therefore, for example, a stent formed into a spiral and approximately tubular body which is provided in the aneurysm embolization device 30 shown in FIG. 3 can be also used in the same way as in the above-described second embodiment.

It is also desirable that the above-described stent be attached to at least one end of the balloon plane part 2.

As mentioned above, despite such a simple structure, another end of the balloon plane part 2 deploys along the bloodstream and can ensure stable placement of the aneurysm embolization device in the blood vessel without displacement.

Furthermore, when this embodiment includes the means for stabilizing the balloon plane part 2 in a blood vessel through the radial expansion, it is desirable that the means more effectively expand radially. In other words, the above-mentioned approximately tubular stent 3 shown in the second embodiment should autonomously expand with its elastic force at a desirable position. However, if not, and if the stent is sought to expand at a desirable position, additional means is needed to support the expansion of the means for stabilizing the balloon plane part 2 in a blood vessel. Therefore, in this embodiment, the expansion supporting means is further provided.

Such an aneurysm embolization device including the expansion supporting means can be exemplified in FIG. 7.

As shown in FIG. 7, the aneurysm embolization device further includes the expansion balloon 4 which is expandable inside the stent 3. The expansion balloon 4 can push and expand the stent 3 from the inside of the stent 13 by inflating, and can be, for example, a flexible and stretchable bag made of latex, silicone, or the like.

In FIG. 7, the expansion balloon 4 is constituted so that the balloon 4 is inserted and placed into the stent 3 constituted of the approximately tubular body cells 3A and 3B, and that the balloon 4 can be evenly inflated by slowly introducing a liquid, such as isotonic saline, through a liquid-supplying tube 14 thereinto.

According to the aneurysm embolization device with the above-described constitution, radial expansion of the stent 3 in a blood vessel is supported, and the balloon plane part 2 can be stabilized in the blood vessel efficiently and effectively.

Next, description will be given of methods for operating the aneurysm embolization devices with the above-mentioned constitutions.

The aneurysm embolization devices of the present invention can be used for different sites of an aneurysm as appropriate. First of all, description will be given of a case where an aneurysm occurred in a side surface, at a midstream of, a non-branched blood vessel is occluded. In this case, although any of the above-described aneurysm embolization devices can be used, here, an operation method in which an aneurysm is occluded using an aneurysm embolization device 50 shown in the above-described fourth embodiment will be described as an example.

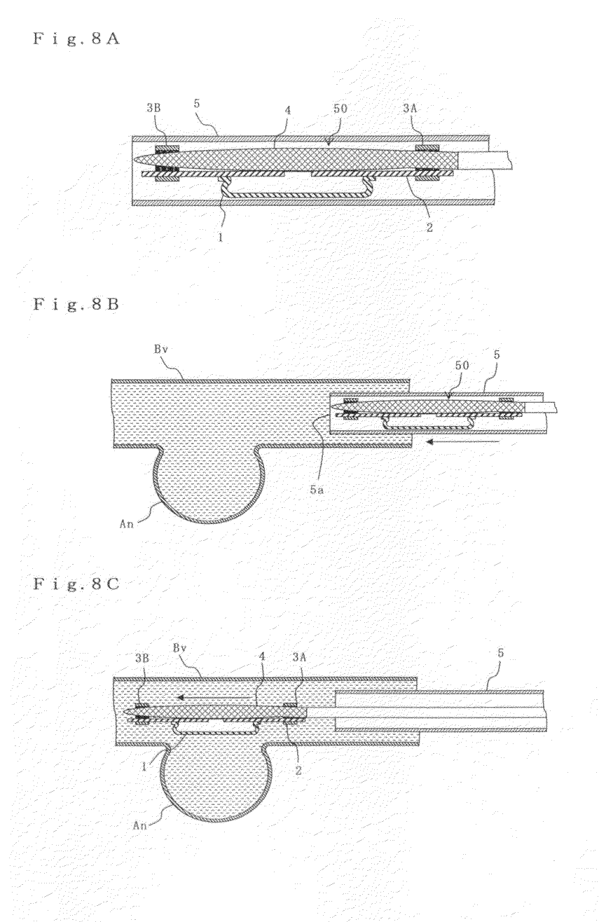

FIGS. 8A to 10B are sectional process drawings for sequentially illustrating a method for operating the aneurysm embolization device 50 shown in the fourth embodiment.

Firstly, as shown in FIG. 8A, the folded aneurysm embolization device 50 according to the fourth embodiment is housed in a lumen of a protective outer sheath member 5 that is formed of a flexible elongated-body having the lumen therein. This aneurysm embolization device 50 includes a balloon dome part 1 and a balloon plane part 2. The balloon plane part 2 further has a stent 3 (3A and 3B) which is formed of an approximately tubular body, and which radially expands in a blood vessel. The aneurysm embolization device 50 further includes an expansion balloon 4 which is expandable inside the stent 3.

It is preferred that such a protective outer sheath member 5 be flexible so that the member can easily follow meanderings of a blood vessel. The protective outer sheath member 5 may be a microcatheter used for cerebrovascular treatment. The microcatheter may be a wire-guided catheter which follows a guide wire having been inserted in a blood vessel ahead of the wire-guided catheter, and which is mainly used for aneurysm embolization. Alternatively, the microcatheter may be a flow-guided catheter which is guided by bloodstream. Examples of materials used for such a catheter include: various thermoplastic resins and thermosetting resins including polyvinyl chloride and polyolefins such as polyethylene, polypropylene, polyurethane, ethylene-propylene copolymers and ethylene-vinyl acetate copolymers, polyesters such as polyethylene terephthalate and polybutylene terephthalate, polyamide, polyimide, polytetrafluoroethylene, polyvinylidene-fluoride, and other fluorocarbon resins; thermoplastic elastomers such as polyamide elastomer and polyester elastomer; various rubbers; and the like.

Meanwhile, it is desired that that the protective outer sheath member (microcatheter) 5, at least a tip part thereof, be formed of a radiopaque material including metals such as platinum, gold, silver and tungsten, and alloys thereof, and that the protective outer sheath member 5 have an imaging ability allowing to monitor the manipulation using radioscopy. This enables a location of the tip part to be monitored during the operation of the protective outer sheath member 5.

Then, as shown in FIG. 8B, the protective outer sheath member 5 housing the aneurysm embolization device 50 is inserted into a parent vessel Bv, and the protective outer sheath member 5 is manipulated to place a tip end part 5a of the protective outer sheath member 5 at a desired position where the aneurysm embolization device 50 is intended to be left. In other words, the tip part 5a of the protective outer sheath member 5 is placed in the vicinity of an aneurysm An. As mentioned above, such a protective outer sheath member 5 can transport the aneurysm embolization device 50 to the vicinity of the aneurysm while housing the aneurysm embolization device 50.

Subsequently, as shown in FIG. 8C, the aneurysm embolization device 50 is ejected from the tip part 5a of the protective outer sheath member 5, and delivered above the aneurysm An. An arrow in the figure indicates a direction in which the aneurysm embolization device 50 is ejected from the protective outer sheath member 5.

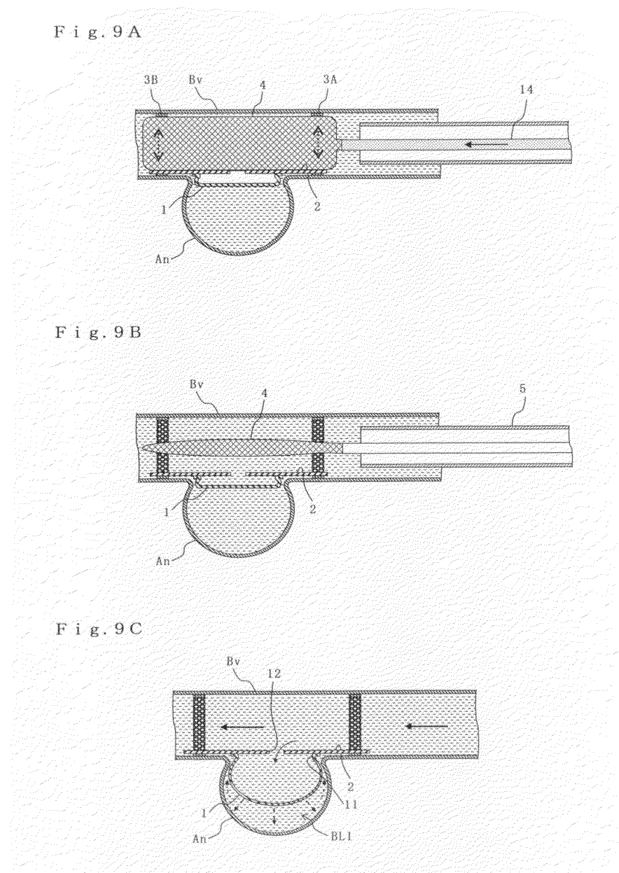

Then, as shown in FIG. 9A, a liquid, for example, isotonic saline is supplied through a liquid-supplying tube 14 into the aneurysm embolization device 50, while the position of the aneurysm embolization device 50 is held the same. The liquid causes the expansion balloon 4 to expand, and then the stent 3 (3A and 3B) to expand. The stent 3 (3A and 3B) is pressed and bonded to the parent vessel Bv, thereby placing the balloon dome part 1 in the aneurysm An, and deploying the balloon plane part 2 so as to cover a mouth part of the aneurysm An. A solid arrow in the figure indicates a direction in which the liquid (saline) is supplied through the liquid-supplying tube 14, and dotted arrows indicate directions in which the stent 3 (3A and 3B) is expanded by inflation of the expansion balloon 4.

As mentioned above, the balloon plane part 2 is placed in the parent vessel to occlude the aneurysm by providing the expansion balloon 4. Accordingly, the aneurysm embolization device 50 is left, so that the balloon dome part 1 is inserted into the aneurysm An, and that the balloon plane part 2 covers the mouth part of the aneurysm An.

Subsequently, after the aneurysm embolization device 50 is stabilized in a blood vessel by the expansion of the stent 3 (3A and 3B), the expansion balloon 4 is deflated as shown in FIG. 9B. The expansion balloon 4 is then slid back from the inside of the stent 3 and removed from the inside of the parent vessel Bv together with the protective outer sheath member 5.

In the above-described manner, the aneurysm embolization device 50 according to the fourth embodiment of the present invention is operated.

As a result, as shown in FIG. 9C, blood streaming in the parent vessel Bv flows into the balloon dome part 1 from a hole 12 of the balloon plane part 2. This blood (hereinafter, referred to as “blood in the balloon dome part”) BL2 expands the balloon dome part 1, and pushes blood having been in the aneurysm An (i.e., a tiny amount of blood remained between the aneurysm An and the balloon dome part 1; hereinafter, referred to as “blood in the aneurysm”) BL1 out of the aneurysm, that is, the parent vessel. Note that, in the figure, thick solid arrows indicate a direction in which blood flows in the parent vessel Bv; a thin solid arrow indicates a direction in which blood flows into the balloon dome part 1 from the parent vessel Bv; and thin dotted arrows indicate directions in which the balloon dome part 1 expands.

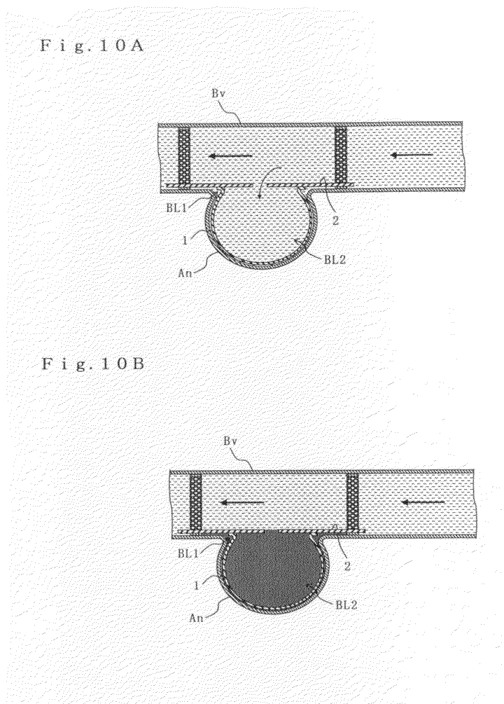

Subsequently, once the inside of the balloon dome part 1 is filled with the flowed blood as shown in FIG. 10A, the stream of the blood in the balloon dome part BL2 is repressed, and the stream of the blood in the aneurysm BL1 is also repressed.

Then, as shown in FIG. 10B, the blood in the balloon dome part BL2 whose stream has been repressed forms a clot, and finally occludes the aneurysm. Moreover, the blood in the aneurysm BL1, which remains between the aneurysm An and the balloon dome part 1, eventually forms a clot, also.

Next, description will be given of a case where an aneurysm occurred at a spot where a blood vessel branches off is occluded. Here, an operation method in which an aneurysm is occluded using an aneurysm embolization device 40 shown in the above-described third embodiment will be described as an example.

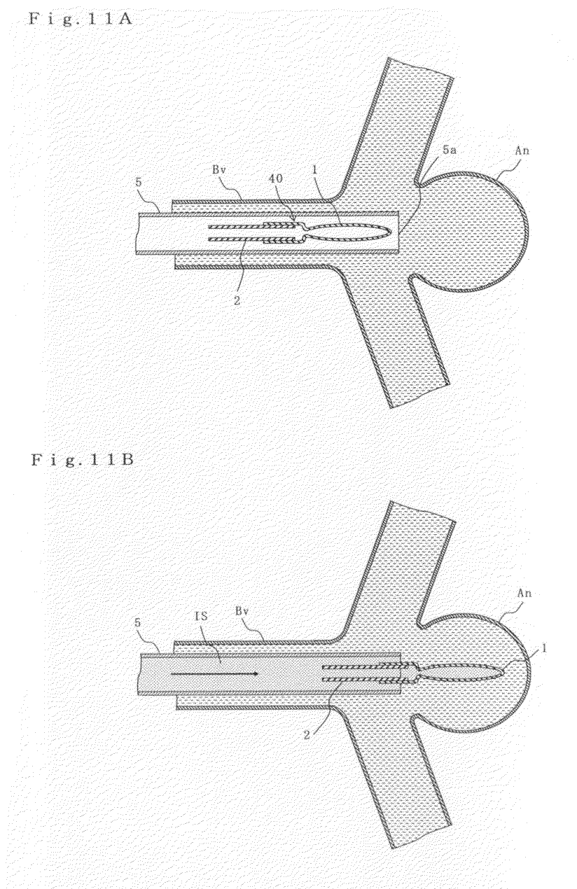

FIGS. 11 A to 12B are sectional process drawings for sequentially illustrating a method for operating the aneurysm embolization device 40 shown in the third embodiment.

Firstly, the folded aneurysm embolization device 40 is housed in a lumen of a protective outer sheath member 5 that is formed of a flexible elongated-body having the lumen therein. This aneurysm embolization device 40 includes a balloon dome part 1 and a balloon plane part 2. It is preferred that such a protective outer sheath member 5 is flexible so that the member can easily follow meanderings of a blood vessel. The protective outer sheath member 5 may be a microcatheter used for cerebrovascular treatment.

Then, as shown in FIG. 11A, the protective outer sheath member 5 housing the aneurysm embolization device 40 is inserted into a parent vessel Bv, and the protective outer sheath member 5 is manipulated to place a tip part 5a of the protective outer sheath member 5 at a desired position where the aneurysm embolization device 40 is intended to be left. In other words, the tip part 5a of the protective outer sheath member 5 is placed in the vicinity of an aneurysm An.

Subsequently, as shown in FIG. 11B, while the position of the tip part 5a of the protective outer sheath member 5 delivered immediately in front of the aneurysm An is held the same, the aneurysm embolization device 40 is ejected by pressure of a liquid, for example, isotonic saline IS which is slowly supplied through the lumen of the protective outer sheath member 5. An arrow in the figure indicates a direction in which the pressuring liquid IS, which presses out the aneurysm embolization device 40 is supplied. As a result, the balloon dome part 1 is firstly pushed out, then the balloon dome part 1 expands and is stabilized in the aneurysm An. Moreover, the expanded balloon dome part 1 gives a force to expand the balloon plane part 2, which is ejected following the balloon dome part 1, in all the directions.

When the aneurysm embolization device 40 is further pushed out slowly by the pressure of the isotonic saline IS, the balloon plane part 2 deploys and stably covers a mouth part of the aneurysm An.

Then, after the above process is completed, the protective outer sheath member 5 is slid back and removed from the inside of the parent vessel Bv.

In the above-described manner, the aneurysm embolization device 40 according to the third embodiment of the present invention is operated.

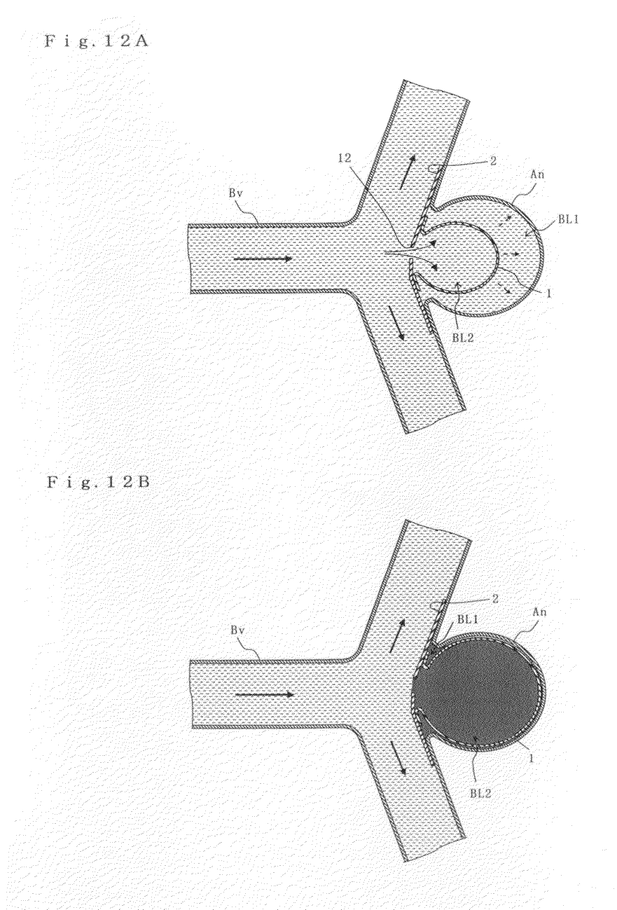

As a result, as shown in FIG. 12A, blood streaming in the parent vessel Bv flows into the balloon dome part 1 from the hole 12 of the balloon plane part 2. This blood (hereinafter, referred to as “blood in the balloon dome part”) BL2 expands the balloon dome part 1, and pushes blood having been in the aneurysm An (i.e., a tiny amount of blood left between the aneurysm An and the balloon dome part 1; hereinafter, referred to as “blood in the aneurysm”) BL1 out of the aneurysm, that is, the parent vessel,. Note that, in the figure, thick solid arrows indicate a direction in which the blood flows in the parent vessel Bv; a thin solid arrow indicates a direction in which blood flows into the balloon dome part 1 from the parent vessel Bv; and thin dotted arrows indicate directions in which the balloon dome part 1 expands.

After that, once the inside of the balloon dome part 1 is filled with the flowed blood as shown in FIG. 12B, the stream of the blood in the balloon dome part BL2, which has flowed into the balloon dome part 1, is repressed. Then, the blood in the balloon dome part BL2 forms a clot, and finally occludes the aneurysm. Moreover, the stream of the blood in the aneurysm BL1, which remains between the aneurysm An and the balloon dome part 1, is also repressed, and thus the blood in the aneurysm BL1 eventually forms a clot, as well.

In this occlusion process, because of the clot formation by the blood in the balloon dome part BL2, the balloon dome part 1 prevents the aneurysm embolization device 40 from being swept away by the blood flow, thus functioning as an anchor member to aid remaining of the aneurysm embolization device at the site of the aneurysm. In addition, the clot formed by the blood in the aneurysm BL1 functions as a paste member to adhere the inner wall of the aneurysm An to the outer surface of the balloon dome part 1 as well as the inner wall of the parent vessel Bv to the other side 2b of the balloon plane part 2. This prevents the aneurysm embolization device 40 from being easily detached from the aneurysm.

As mentioned above, by using the aneurysm embolization device of the present invention, it is possible to treat an aneurysm in a blood vessel by occluding the aneurysm readily and reliably in an appropriate manner according to the site, the shape of a neck part, and the size of the aneurysm.

### INDUSTRIAL APPLICABILITY

The present invention is industrially useful in the field where aneurysm embolization devices are handled to treat an aneurysm in a blood vessel. The invention is particularly useful for a market of aneurysm embolization devices to treat an aneurysm in the brain by occluding the aneurysm.

What is claimed is:

1. An aneurysm embolization device for treating an aneurysm in a blood vessel by occluding the aneurysm, comprising at least:
a sac-shaped balloon dome part which is inserted into the aneurysm, then expanded and left therein; and
a balloon plane part which is provided at an opening of the balloon dome part and covers a mouth part of the aneurysm,
wherein the balloon plane part includes a hole communicating with an inside of the balloon dome part.

2. The aneurysm embolization device according to claim 1, wherein the balloon plane part further includes an anchor member (stent) which is expanded and left in the blood vessel.

3. The aneurysm embolization device according to claim 2, wherein the anchor member is attached to at least one end of the balloon plane part.

4. A method for operating an aneurysm embolization device for treating an aneurysm in a blood vessel by occluding the aneurysm, the aneurysm embolization device including at least: a sac-shaped balloon dome part which is inserted into the aneurysm, then expanded and left therein; and a balloon plane part which is provided at an opening of the balloon dome part and covers a mouth part of the aneurysm, the balloon plane part having a hole communicating with an inside of the balloon dome part, the method comprising the successive steps of:
α1) housing the aneurysm embolization device into a lumen of a protective outer sheath member formed of a flexible elongated-body having the lumen therein;
α2) manipulating the protective outer sheath member to place a tip part of the protective outer sheath member at a desired position where the aneurysm embolization device is intended to be left; and
α3) ejecting the aneurysm embolization device from the tip part of the protective outer sheath member, placing the balloon dome part at the desired position, and then, deploying the balloon plane part to cover the desired position.

5. The method for operating an aneurysm embolization device according to claim 4,
wherein, in the step α3, the aneurysm embolization device is ejected by pressure of a liquid which is supplied through an inside of the lumen of the protective outer sheath member.

6. A method for operating an aneurysm embolization device for treating an aneurysm in a blood vessel by occluding the aneurysm, the aneurysm embolization device including at least: a sac-shaped balloon dome part which is inserted into the aneurysm, then expanded and left therein; and a balloon plane part which is provided at an opening of the balloon dome part and covers a mouth part of the aneurysm, the balloon plane part having a hole communicating with an inside of the balloon dome part, the balloon plane part further including an anchor member (stent) which is expanded and left in the blood vessel and an expansion balloon which supports the expansion of the anchor member, the method comprising the successive steps of:
β1) housing the aneurysm embolization device into a lumen of a protective outer sheath member formed of a flexible elongated-body having the lumen therein;
β2) manipulating the protective outer sheath member to place a tip part of the protective outer sheath member at a desired position where the aneurysm embolization device is intended to be left;
β3) ejecting the aneurysm embolization device from the tip part of the protective outer sheath member, expanding the anchor member by inflating the expansion balloon, placing the balloon dome part at the desired position, and then, deploying the balloon plane part to cover the desired position; and
β4) deflating the expansion balloon and then sliding back the expansion balloon from an inside of the anchor member.
